(19) United States
(12) Patent Application Publication
(10) Pub. No.: US 2003/0141178 A1
(43) Pub. Date: Jul. 31, 2003

(54) Energizing gas for substrate processing with shockwaves

(75) Inventors: Hongqing Shan, Cupertino, CA (US); Joseph Bach, Morgan Hill, CA (US)

Correspondence Address: APPLIED MATERIALS, INC., 2881 SCOTT BLVD. M/S 2061, SANTA CLARA, CA, 95050, US

(73) Assignee: Applied Materials, Inc.

(21) Appl. No.: 10/061,134

(22) Filed: Jan. 30, 2002

(51) Int. Cl. C07C 1/00; C07C 2/00; C07C 4/00; C07C 5/00

(52) U.S. Cl. 204/157.15; 422/186

(57) ABSTRACT

A substrate processing chamber has a substrate support to support a substrate in the housing. A shockwave gas energizer is provided to generate shockwaves to at least partially energize a process gas and provide the energized process gas into the housing. In one version, the shockwave gas energizer comprises a gas nozzle adapted to accelerate the process gas and a gas flow blocker to obstruct the accelerated flow of the process gas emanating from the gas nozzle to generate the shockwaves. The chamber also has an exhaust to exhaust the process gas from the housing.

### BACKGROUND

Embodiments of the present invention relate to energizing gas for substrate processing.

In the fabrication of electronic components, such as integrated circuits and flat panel displays, semiconductor, dielectric and conductor materials, for example, polysilicon, silicon dioxide, and metal containing materials, respectively, are formed on a substrate. Some of these materials are deposited by chemical vapor deposition (CVD) or physical vapor deposition (PVD) processes, and others may be formed by oxidation or nitridation of substrate materials. For example, in chemical vapor deposition processes, a deposition gas is introduced into the chamber and energized to deposit a film on the substrate. In physical vapor deposition, a target of sputtering material is sputtered by an energized gas to deposit a layer of the target material on the substrate. In subsequent etching processes, a patterned mask comprising a photoresist or hard mask material is formed on the substrate material by lithography, and the portions of the substrate material that are exposed between the mask features are etched by an energized gas, such as a halogen-containing or oxygen-containing gas. The deposition, etching, and other processes such as planarization processes, are conducted in sequence, to process the substrate to fabricate integrated circuits and other electronic devices.

In such processes, gas energizers are used to energize the gas by coupling microwaves or RF energy to the gas. For example, electrodes or an inductor antenna may be used to capacitively or inductively, respectively, couple RF energy to the gas. The coupled energy ionizes or dissociates the gas to form energized ionized or dissociated gas species that process the substrate.

However, conventional gas energizers have certain limitations. For example, it is often difficult to energize the process gas to preferentially generate certain types of gaseous species, such as for example, to preferentially form ionized species, non-ionized molecular free radicals, or non-ionized atomic free radicals, within the energized process gas. It may be desirable to preferentially dissociate or ionize the process gas to better control, for example, the etching parameters of an etching process, such as etching selectivity ratios, or the deposition of material in a deposition process. For example, in etching processes, it may be desirable to etch the substrate with an etchant gas that contains a higher percentage of, for example, dissociated species than ionized species, or vice versa. It may also be desirable to selectively dissociate or ionize certain gas components that are more reactive to the material being etched than other gas components. In deposition processes, it may also be desirable to better control the dissociation or ionization efficiency of particular components of the process gas to obtain a desired stoichiometry, crystalline grain size, or other such properties of the deposited material; or to control other deposition process characteristics, such as for example, to improve the rate of deposition on the sidewalls of trenches in the substrate relative to the rate of deposition of material on other portions of the substrate.

Thus it is desirable to controllably energize a process gas to provide better control of the substrate processing parameters. It is also sometimes desirable to preferentially dissociate or ionize the process gas. It may further be desirable to selectively energize a predefined component of the process gas over the other gas components.

### SUMMARY

A substrate processing chamber comprises a housing; a substrate support to support a substrate in the housing; a shockwave gas energizer to generate shockwaves in a process gas to at least partially energize the process gas and provide the energized process gas into the housing to process the substrate; and a gas exhaust to exhaust the process gas from the housing.

A substrate processing method comprises placing a substrate in a process zone; generating shockwaves in a process gas to at least partially energize the process gas and providing the energized process gas to the process zone to process the substrate; and exhausting the process gas from the process zone.

A substrate processing chamber comprises a housing; a substrate support to support a substrate in the housing; a shockwave gas energizer comprising: (i) a gas nozzle to provide an accelerated flow of process gas, the gas nozzle comprising a gas inlet to receive a process gas, a gas outlet to eject the process gas, and a flow constricting throat between the gas inlet and the gas outlet; (ii) a gas flow blocker to obstruct the accelerated flow of the process gas to generate shockwaves in the process gas that at least partially energize the process gas; and (iii) an aperture to provide the energized process gas into the housing; and a gas exhaust to exhaust the process gas from the housing.

A substrate processing chamber comprises a housing; a substrate support to support a substrate in the housing; means for generating shockwaves in a process gas to at least partially energize the process gas, and provide the energized process gas into the housing to process the substrate; and an exhaust to exhaust the process gas.

A substrate processing chamber comprises a housing; a substrate support to support a substrate in the housing; two shockwave gas energizers positioned to face each other and direct an energized process gas toward a process zone between the two shockwave gas energizers, each shockwave gas energizer comprising: (i) a gas nozzle to provide an accelerated flow of process gas, the gas nozzle comprising a gas inlet to receive a process gas, a gas outlet to eject the process gas, and a flow constricting throat between the gas inlet and the gas outlet; (ii) a gas flow blocker to obstruct the accelerated flow of the process gas to generate shockwaves in the process gas that at least partially energize the process gas; (iii) a gas guide extending below the gas flow blocker such that solid particles from the gas nozzle fall down onto the gas guide rather than onto the substrate; and (iv) an aperture to provide the energized process gas into the housing; and a gas exhaust to exhaust the process gas from the housing.

### DRAWINGS

These features, aspects, and advantages of the present invention will become better understood with regard to the following description, appended claims, and accompanying drawings which illustrate exemplary features of the invention. However, it is to be understood that each of the features can be used in the invention in general, not merely in the context of the particular drawings, and the invention includes any combination of these features, where:

FIG. 1 is a schematic sectional side view of an embodiment of a substrate processing chamber;

FIG. 2 is a sectional side view of the gas energizer of the chamber of FIG. 1;

FIG. 3 is a sectional side view of a gas energizer having a gas flow blocker with a convex surface;

FIG. 4 is a sectional side view of a gas energizer having a gas flow blocker with a concave surface;

FIG. 5 is a schematic sectional side view of an embodiment of a substrate processing chamber; and

FIG. 6 is a schematic block diagram of the controller of the chamber of FIG. 1 showing the hierarchical structure of the software code that operates the controller.

### DESCRIPTION

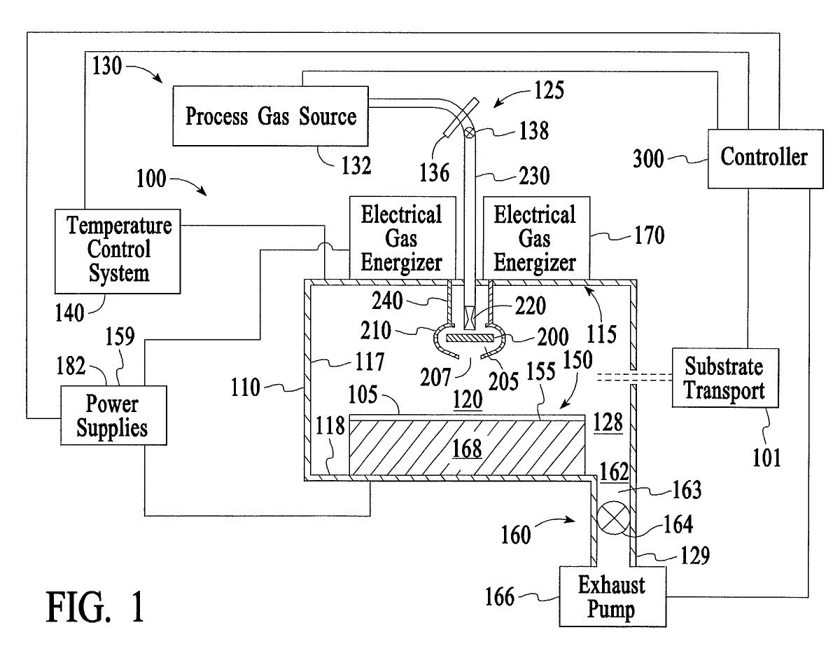

A substrate processing chamber 100 that is useful for processing a substrate 105 to fabricate electronic circuits on the substrate 105 is shown in FIG. 1. The substrate processing chamber 100 may be used to etch regions of, or deposit material on, the substrate 105. For example, the substrate processing chamber 100 may be used for deposition processes such as chemical vapor deposition (CVD) or physical vapor deposition (PVD) to deposit a layer of material on the substrate 105. Generally, the substrate processing chamber 100 is mounted on a platform (not shown) that provides electrical, plumbing, and other support functions to the chamber 100 as well as other chambers. The chamber 100 comprises a housing 110 comprising a ceiling 115, sidewall 117, and bottom wall 118, which are typically fabricated from metal or ceramic materials. The housing 110 defines a process zone 120 and encloses a substrate support 150 having a substrate receiving surface 155. To process the substrate 105, the housing 110 of the chamber 100 is evacuated and maintained at a predetermined sub-atmospheric pressure. In one embodiment, the process zone 120 of the housing 110 is maintained at a pressure of less than about 1 Torr, or even less than about 500 mTorr or even from 10 to 50 mTorr. A substrate 105 is transported into the process zone 120 and placed on the substrate receiving surface 155 of the support 150, such as by a substrate transport 101.

The substrate processing chamber 100 comprises a shockwave gas energizer 125 to at least partially energize a process gas by inducing compression shockwaves in a volume of the process gas, and provide the energized process gas into the housing 110. The shockwave gas energizer 125 may be adapted to preferentially energize a predefined component of the process gas, or to selectively dissociate or ionize the process gas to preferentially produce excess ionized or non-ionized species, or relatively more molecular free radicals or atomic free radicals. For example, the shockwave gas energizer 125 may be capable of generating and sustaining a plasma of the process gas. The shockwave gas energizer 125 operates by generating shockwaves in a flowing or stationary volume of the gas, the intensity, frequency, and other such properties of the shockwaves being selected to achieve the desired result. In one embodiment, the shockwave gas energizer 125 is mounted in the housing 110 in opposing relationship to the substrate support 150.

Generally, the shockwave gas energizer 125 operates by accelerating a flow of process gas to a sufficiently high velocity, and thereafter, blocking or otherwise obstructing the accelerated gas flow to generate shockwaves through a volume of the gas. A gas supply 130 provides the process gas for the shockwave gas energizer 125 from a gas source 132. The gas source 132 provides process gas at desirable pressures to the shockwave gas energizer 125 to allow the energizing of the process gas therein by the creation of shockwaves. For example, the ratio of the pressure of the process gas provided by the gas source 132 to the pressure of the gas in the housing 110 may be selected to be at least about 10 to 1. In one embodiment, the ratio of pressure of the gas in the gas source 132 to the pressure in the process chamber 100 is at least about 2000 to 1, or even at least about 50,000 to 1. For example, the gas source 132 can provide process gas at a pressure of at least about 600 Torr, at least about 760 Torr, or even at least about 7600 Torr. The gas supply 130 comprises one or more gas conduits 230 having gas flow control valves 136 thereon, such as mass flow controllers, to adjust the flow of the process gas components flowing through the gas conduits 230. The gas flow control valves 136 may be electro-mechanically controlled, such as using an electromagnet, or simply mechanically controlled. A flow meter 138 may also be provided to determine or control the flow of process gas through the gas conduit 230.

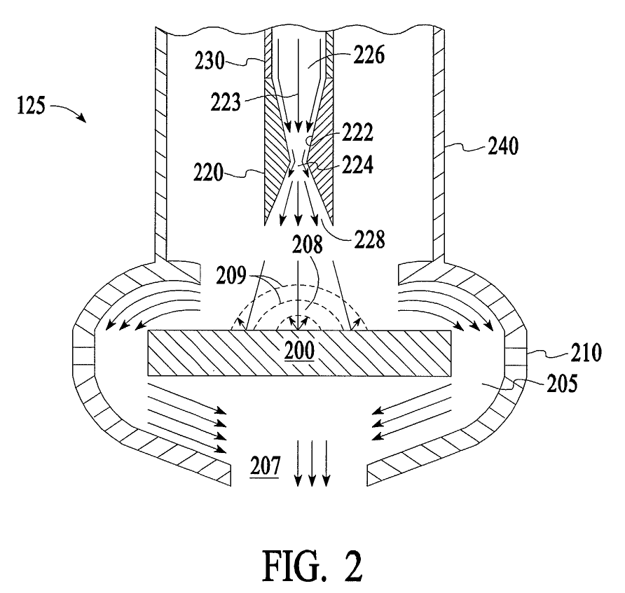

The shockwave gas energizer 125 comprises a gas nozzle 220 that is adapted to accelerate the pressurized process gas to a velocity that is sufficiently high to allow the formation of shockwaves that energize the process gas by dissociating the gas. For example, the gas nozzle 220 may accelerate the process gas to a velocity of at least about Mach 1. In one embodiment, the gas nozzle 220 accelerates the process gas to a velocity of from about Mach 1 to about Mach 5, such as from about Mach 2 to about Mach 3. In one version, as illustrated in FIG. 2, the gas nozzle 220 is shaped to accelerate the process gas as it passes through the nozzle 220. The gas nozzle 220 comprises a gas inlet 226 to receive a process gas, a gas outlet 228 to eject the process gas, and a flow constricting throat 224 between the gas inlet 226 and the gas outlet 228. In this version, the gas nozzle 220 comprises a wall 222 that tapers radially inwardly from the gas inlet 226 to the flow constricting throat and tapers radially outwardly from the flow constricting throat 224 to the gas outlet 228. The gas inlet 226 comprises a first diameter and the constricting portion 224 comprises a second diameter. In one embodiment, the ratio of the first to the second diameter is at least about 5:1. The gas outlet 228 comprises a third diameter, and the ratio of the third to the second diameter may be at least about 10:1. The increasing diameter between the flow constricting throat 224 and the gas outlet 228 increases the gas velocity and ejects the process gas at a high velocity from the gas outlet 228. As the process gas leaves the constricting portion 224, it encounters a region having a pressure value that is between the pressure in the gas supply 132 and a pressure in the vacuum chamber 100, and thus begins to expand. In this manner, the process gas is accelerated down the path of the nozzle 220. At the gas outlet 228, the process gas encounters the low-pressure environment of the vacuum chamber 100, and is still further accelerated. The gas nozzle 220 may comprise an adjustable band, collar, or ring (not shown) surrounding the passage to constrict the passage. A suitable gas nozzle 220 is a venturi nozzle, such as that available, for example, from Dosch Messapparate GmbH, Berlin, Germany.

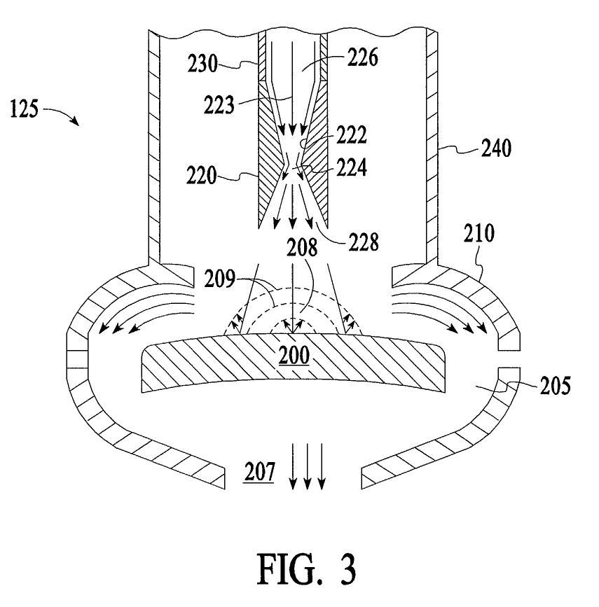

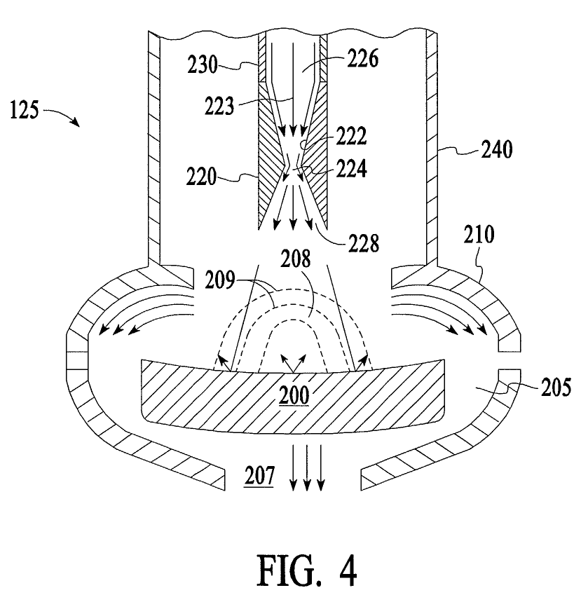

The shockwave gas energizer 125 further comprises a gas flow blocker 200 to obstruct and rapidly decelerate the process gas after it is ejected from the gas outlet 228 of the gas nozzle 220 to allow the generation of shockwaves 209 in the process gas. These shockwaves 209 dissociate or ionize a volume of flowing or non-flowing gas in a dissociation region 208 near or about the gas flow blocker 200, where the gas accumulates at high pressure. When the process gas collides with the gas flow blocker 200, some of the translational kinetic energy of the process gas molecules is converted into thermal energy, which can cause collisions between particles, breaking of molecular bonds, and excitation of electrons into higher states of potential energy. In one version, the gas flow blocker 200 comprises a flat plate (as shown). In another version, as shown in FIG. 3, the gas flow blocker 200 comprises a convex surface having an apex about the principal direction of flow of the incident process gas. As the process gas impacts the gas flow blocker 200, the convexity of the gas flow blocker 200 guides the process gas past the gas flow blocker 200 along an edge of the gas flow blocker 200. In yet another version, as shown in FIG. 4, the gas flow blocker 200 comprises a concave surface having an apex about the principal direction of flow of the incident process gas. For example, the gas flow blocker 200 may comprise a bowl or V-shaped platter. As the process gas impacts the concave gas flow blocker 200, the concavity of the gas flow blocker 200 serves to temporarily trap the rapidly flowing process gas in a containment region in front of the gas flow blocker 200. In one embodiment, the gas flow blocker 200 is oriented perpendicularly to a principal flow direction 223 of the process gas from the gas nozzle 220. The gas flow blocker 200 may also have a superficial obstruction area that is adapted to selectively energize a predefined component of the process gas. The gas flow blocker 200 may be supported by the housing 110, such as by the ceiling 115 of the housing 110, by a holder 240.

The gas nozzle 220 and gas flow blocker 200 of the shockwave gas energizer 125 cooperate to generate the shockwaves 209 that selectively energize and dissociate the process gas. As the rapidly flowing process gas is suddenly blocked, the kinetic energy of the flowing gas molecules is translated into bursts of high-energy, high-pressure compressional shockwaves that thermally excite and selectively energize components of the process gas. For example, the shockwave gas energizer 125 may be adapted to generate a desired rate of dissociation or ionization of the process gas, or to energize particular gas components preferentially over other gas components. For example, the shockwave gas energizer 125 may be adapted to generate particular radical groups. The shockwave gas energizer 125 may also be adapted to selectively dissociate the process gas into either atomic or molecular radicals. In one embodiment, it is desirable to increase the rate of generation of molecular uncharged free radicals, without correspondingly increasing the rate of generation of atomic uncharged free radicals, to improve an etching selectivity of the process gas, such as to decrease an etching ratio of a mask or photoresist overlying a substrate to the underlying substrate itself. For example, when using C₄F₆ as the process gas, it may be desirable to limit the formation of atomic F radicals while assisting the formation of certain molecular free radicals that derive from the C₄F₆.

In one version, the gas nozzle 220 and the gas flow blocker 200 are spaced apart by a distance that is selected to desirably generate shockwaves to energize the process gas. The distance between the gas nozzle 220 and the gas flow blocker 200 affects the spatial density and/or velocity distribution of the process gas at the gas flow blocker 200. For example, a small distance of less than about 1 cm between the gas nozzle 220 and the gas flow blocker 200 may be selected so that the process gas impinges on the gas flow blocker 200 at a high velocity to generate shockwaves of sufficient intensity to yield a desired rate of gas dissociation. In one embodiment, the gas nozzle 220 conveys the process gas from near the ceiling 115 of the housing 110 down to the gas flow blocker 200 such that the process gas impacts the gas flow blocker 200 to generate shockwaves that energize the process gas, then passes down toward the substrate 105.

In capacitively or inductively generated electron impact dissociation conventionally used in plasma processing chambers, atomic free radicals are generated and ionized in undesirably large proportions. In the present invention, on the other hand, the shockwave induced thermal dissociation tends to break long chained species into smaller molecular free radicals. For example, C₄F₆ is broken into C₂F₃, CF₂, C₂F₂, etc. without excessive dissociation into F or excessive ionization. Thus, the shockwave gas energizer 125 may dissociate process gas components without excessive ionization of the process gas to enhance etching selectivity.

The shockwave gas energizer 125 may further comprise channels 205 to convey the energized process gas away from the gas flow blocker 200 and toward the process zone 120. For example, the channels 205 may comprise passages between the gas flow blocker 200 and a gas guide 210 that serves to form the channels 205 (as shown), or between the gas flow blocker 200 and an interior wall 115, 117, 118 of the housing 110 (not shown). The gas guide 210 may be attached to the housing 110 by the holder 240. In one version, the channels 205 are of a length adapted to minimize recombination of the free molecular radicals, such as of less than about 10 mm. Additionally, the channels 205 may comprise a material that is resistant to being corroded by the energized process gas. For example, the channels 205 may comprise a polymer, such as Teflon, a ceramic material, such as Al₂O₃, SiC, SiN, or AlN, or a metal, such as Al. The channels 205 terminate in apertures 207 that direct the energized gas into the housing I 10. In one version, the gas nozzle 220, gas flow blocker 200, channels 205, and apertures 207 are adapted to control a spatial energy distribution of the process gas. In one embodiment, it is desirable to provide a plasma immediately above the substrate 105. For example, by positioning the gas flow blocker 200 near the substrate 105 and orienting the gas nozzle 220 toward the gas flow blocker 200 to desirably energize the process gas, a plasma sheath having a controlled composition of gas species can be provided immediately above the substrate 105.

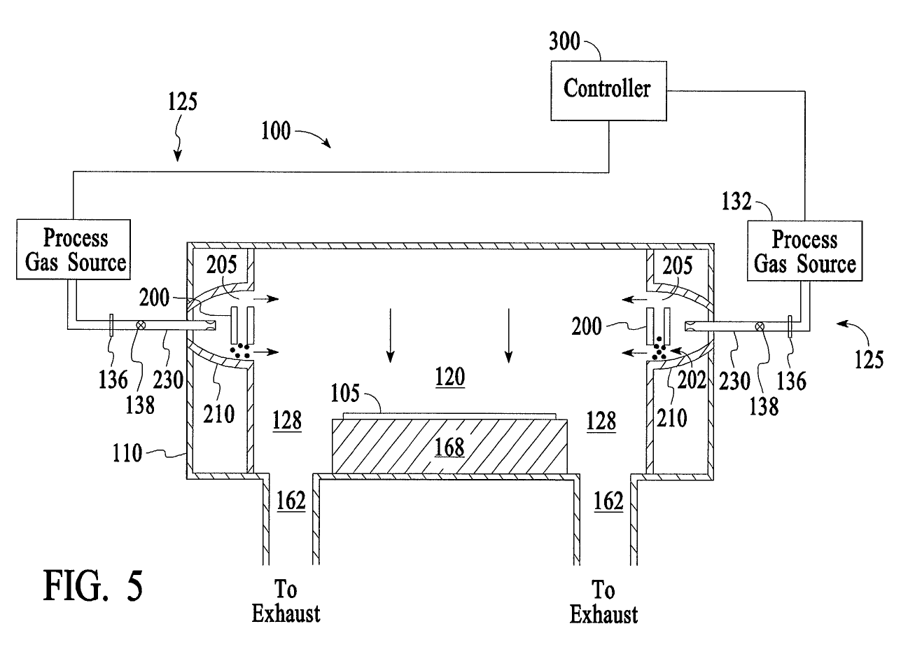

In one version, as illustrated in FIG. 5, the shockwave gas energizer 125 comprises a gas nozzle 220 and gas flow blocker 200 that are positioned such that solid particles 202 from the nozzle 220 do not contaminate the substrate 105. For example, a part of the gas guide 210 may be extend below the gas flow blocker 200 so that the solid particles 202 fall down onto the gas guide 210 rather than onto the substrate 105. Meanwhile, the energized gas passes along the channels 205 and toward the process zone 120 above the substrate 105 to process the substrate 105. In one embodiment, the chamber 100 comprises a set of two shockwave gas energizers 125. For example, the shockwave gas energizers 125 may be positioned to face each other. Thus, the shockwave gas energizers 125 direct the energized process gas toward the process zone 120 between the two gas energizers 125 in order for the energized process gas to be concentrated at the process zone 120 and effectively process the substrate 105.

Returning to FIG. 1, the gas source 132 provides a process gas having process gas components that can be mixed to provide a predetermined composition that, for example, may be suitable for etching material from a substrate 105 or depositing a layer of material on a substrate 105. Thus, the process gas may include a single gas component or maybe a mixture of gas components. For example, in one embodiment, the process gas comprises a non-reactive gas and a reactive gas. The non-reactive gas may be a diluent or inert gas, such as argon, that serves to contain the reactive gas above the substrate 105 without reacting with the gas or substrate 105, or that acts as a diluent to energize and promote reaction of the reactive gas with the substrate. The diluent gas may be provided to facilitate the reaction by, for example, colliding with the energized gas molecules to strip away electrons and form other energized gas species. The diluent gas may also be provided to reduce the resident time of the reactive gas in the chamber 100. If the chamber 100 is used for etching, the reactive gas comprises an etching gas suitable for etching material on the substrate 105. As an example, in the etching of silicon containing substrate material, the reactive gas may comprise a diluent gas such as nitrogen or argon; and the second process gas may comprise a reactive gas such as a halogen containing gas, such as for example, Cl₂, BCl₃, HCl, F₂, CHF₃, C₄F₆, CF₄, C₅F₈, and equivalents thereof another embodiment, if the chamber 100 is used for deposition, the reactive gas comprises a deposition gas suitable for depositing material on the substrate 105. For example, if the chamber 100 is used for chemical vapor deposition (CVD), a non-reactive gas may be argon, and a deposition gas may be a mixture of reactive gases, which can deposit material on the substrate 105. For example, the deposition gas may be a gas suitable to deposit a metal. In one embodiment, tungsten is deposited by introducing WF₆, argon, and H₂. In one version, additional assistant gases are introduced into the flow of the process gas, such as to assist in energizing the process gas. The assistant gases may be atomic gases to improve the dissociation of the process gas. The atomic assistant gases may transfer their kinetic energies to the process gas via collisions because the atomic assistant gases are non-molecular and thus do not dissociate. In one embodiment, Ar or He is introduced into the process gas to increase the amount of dissociation of the process gas.

The gas source 132 may be a cleaning gas supply (not shown) that provides a cleaning gas into the chamber 100 that may be energized to clean off process residues from surfaces inside the chamber 100. The energized cleaning gas may be, for example, NF₃ or CF₄, which may be energized by microwaves or RF energy in a remote chamber before it is introduced into the chamber 100, or may also be energized by the shockwave gas energizer 125. The cleaning gas is passed through a gas conduit 230 that feeds into the housing 110, such as from above the substrate 105. It may be desirable to provide the energized cleaning gas in contaminated regions of the housing 110 where undesirable process residues tend to deposit. For example, by positioning the gas flow blocker 200 near these regions and orienting and sizing the channels 205 to convey the cleaning plasma toward the regions, pockets of cleaning gas can be provided in these contaminated regions. Additionally, by selectively providing the cleaning gas in these regions, deterioration of the inside of the housing 110 by the cleaning gas in other substantially uncontaminated regions can be avoided.

The substrate processing chamber 100 may also have an electrical gas energizer 170 to further energize the process gas to process the substrate 105 by coupling electromagnetic waves to the process gas. The electrical gas energizer 170 energizes the process gas in the process zone 120 of the housing 110 (as shown) or in a remote zone (not shown) upstream from the housing 110. The electrical gas energizer 170 may comprise, for example, inductive energizer components such as an inductor antenna (not shown) to inductively couple energy to the process gas, or capacitive energizer components such as a pair of electrodes to capacitively couple energy to the process gas, such as a combination of electromagnetic energy and a low-frequency magnetic field of less than about 1000 Hz.

The inductive energizer components energize the process gas by generating a fluctuating magnetic field in the housing 110. The inductive energizer components may comprise an antenna (not shown) to inductively couple magnetic field energy to the process gas. The antenna may include coils having a circular symmetry with a vertical central axis of the chamber 100. In one embodiment, the antenna is adjacent to the ceiling 115 of the housing 110. An antenna power supply 159 provides power to the antenna, for example RF power at a frequency of typically from about 50 kHz to about 600 MHz, and more typically about 13.56 MHz; and at a power level of from about 25 to about 5000 Watts. In another version, the inductive energizer components comprise a microwave source and waveguide (not shown) to activate the process gas by conveying microwave energy into the process gas, such as in a remote chamber (not shown).

The capacitive energizer components, on the other hand, energize the process gas by generating an electric field in the housing 110. The capacitive energizer components comprise two or more process electrodes (not shown) that are maintained at different electric potentials by an electrode power supply 182 to generate an electric field between the process electrodes. Typically, an AC voltage is applied to at least one of the electrodes to generate a fluctuating electric field. In one embodiment, the process electrodes include a first electrode formed by a wall, such as a sidewall 117 or ceiling 115 of the housing 110, that is capacitively coupled to a second electrode formed by the support 150 below the substrate 105. The second electrode is typically fabricated from a metal such as Al, tungsten, tantalum, or molybdenum, and is covered by or embedded in a dielectric. The second electrode may also serve as an electrostatic chuck 168 that generates an electrostatic charge for electrostatically holding the substrate 105 to the receiving surface 155 of the support 150. An electrode power supply 182 may comprise one or more DC or AC voltage supplies to apply the electric potentials to the process electrodes. For example, the electrode power supply 182 may supply an RF voltage to the process electrodes of from about 50 kHz to about 600 MHz and a power level of from about 25 Watts to about 5000 Watts.

In one embodiment, the ceiling 115 comprises a semiconductor material that is sufficiently electrically conductive to be biased or grounded as an electrode to form an electric field in the housing 110 yet provides low impedance to an RF induction field transmitted by the antenna (not shown) above the ceiling 115. A suitable semiconductor material comprises semiconducting silicon having a resistivity of less than about 500 Ω-cm at room temperature.

In one version, the shockwave gas energizer 125 initially generates a process gas plasma, and then the electrical gas energizer 170 is used to maintain the process gas in the plasma phase. This version efficiently takes advantage of the inherent potential energy of the process gas in the gas source 132, which is conventionally wasted. Also, by using the electrical gas energizer 170 to maintain the plasma, the electrical gas energizer 170 can tune specific characteristics of the plasma. For example, the operator may tune power levels of the electrical gas energizer 170 to change plasma characteristics, such as to change a ratio of the process gas that is in the plasma phase to the process gas that is in the gas phase. In another embodiment, a position of the antenna or an electrode can be adjusted to modify process gas characteristics, such as to change a spatial energy distribution of the process gas.

In another version, the shockwave gas energizer 125 and the electrical gas energizer 170 have respective power levels that are tuned to control a ratio of dissociated process gas to ionized process gas. For example, the shockwave gas energizer 125 may primarily dissociate the process gas while the electrical gas energizer 170 primarily ionizes the process gas, so that a ratio of power applied to the shockwave gas energizer 125 to power applied to the electrical gas energizer 170 is selected to set the ratio of dissociated gas to ionized gas.

Gas in the housing 110, such as comprising spent process gas and process byproducts, is exhausted from the housing 110 via a gas exhaust 160 comprising an exhaust zone 128 about an exhaust conduit 162 that has one or more exhaust ports 163. The exhaust zone 128 opens to an exhaust line 129 having a throttle valve 164 to control the pressure of gas in the chamber 100, and one or more exhaust pumps 166 that typically include roughing and high vacuum-type pumps.

The chamber 100 may further comprise a temperature control system 140 to regulate the temperature at one or more sections of the process chamber 100. For example, if the ceiling 115 is made of a semiconductor, the temperature control system 140 may hold the temperature of the ceiling 115 in a range of temperatures at which the semiconductor material provides semiconducting properties and in which a carrier electron concentration is fairly constant with respect to temperature. If the semiconductor is silicon, the temperature range may be from about 100 Kelvin (below which silicon begins to have dielectric properties) to about 600 Kelvin (above which silicon begins to have metallic conductor properties).

In one embodiment, the temperature control system 140 controls the temperature of the ceiling 115 using a plurality of radiant heaters (not shown) such as tungsten halogen lamps or a thermal transfer plate (not shown) made of aluminum or copper, with passages (not shown) for a heat transfer fluid to flow therethrough. A heat transfer fluid source (not shown) supplies heat transfer fluid to the passages to heat or cool the thermal transfer plate as needed to maintain the chamber 100 at a constant temperature. The ceiling 115 is in thermal contact with the thermal transfer plate via a plurality of highly thermally conductive rings (not shown) whose bottom surface rests on the ceiling 115 and whose top surface supports the thermal transfer plate. Positioned around the lower portion of the heat transfer rings may be the inductor antenna. A height of the heat transfer rings is selected so that the thermal transfer plate is supported at a distance above the inductor antenna of at least about one half of the overall height of the antenna. This mitigates or eliminates the reduction in inductive coupling between the antenna and the plasma which would otherwise result from their close proximity to a conductive plane of the thermal transfer plate. In another embodiment, cooling channels (not shown) are provided elsewhere in the process chamber 100 to cool that section of the process chamber 100, such as by flowing a cooling fluid therethrough. If the process chamber 100 is used for deposition, cooling channels (not shown) may be provided in the ceiling 115 to cool the ceiling 115 and to reduce the deposition of material thereon.

Additionally, a support heater (not shown) may heat the support 150 to heat the substrate 105 that is in contact with the support 150. The support heater may comprise, for example, lamps (not shown) that direct radiant energy onto the support 150, or a resistive element (not shown) embedded in the support 150, to heat the support 150 and overlying substrate 105 to suitable temperatures. If the process chamber 100 is used to deposit material on the substrate 105, the support heater may heat the substrate 105 to a temperature sufficiently high to cause the deposition gas to preferentially deposit material on the substrate 105 rather than elsewhere in the housing 110. In another embodiment, cooling channels (not shown) are provided in the substrate support 150 to cool the substrate 105, such as to prevent thermal damage to the substrate 105.

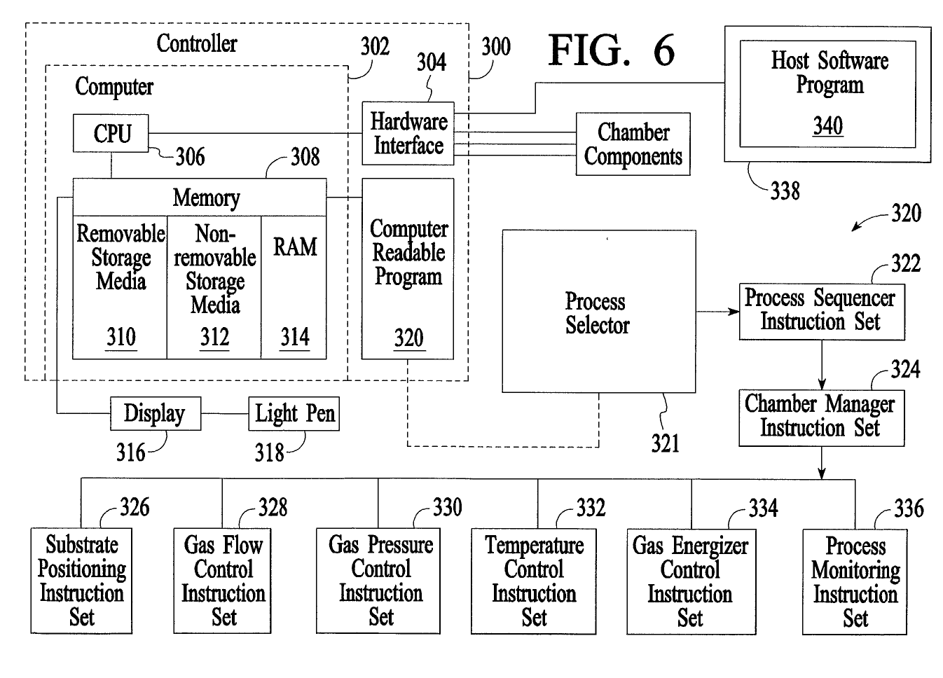

A controller 300, as illustrated in FIG. 6, controls operation of the above-described chamber components to process the substrate 105 in an energized gas. The chamber 100 may be operated by the controller 300 via a hardware interface 304. The controller 300 operates the substrate support 150 to raise and lower the support 150, the gas flow valve 136, the electrical gas energizer 170, and the throttle valve 164, to process the substrate 105 in the energized gas. The controller 300 may comprise a computer 302 which may comprise a central processor unit (CPU) 306, such as for example a 68040 microprocessor, commercially available from Synergy Microsystems, California, or a Pentium Processor commercially available from Intel Corporation, Santa Clara, Calif., that is coupled to a memory 308 and peripheral computer components (not shown). The memory 308 may include removable storage media 310, such as for example a CD or floppy drive, and non-removable storage media 312, such as for example a hard drive, and random access memory 314. The controller 300 may further comprise a plurality of interface cards (not shown) including, for example, analog and digital input and output boards, interface boards, and motor controller boards. The interface between an operator and the controller 300 can be, for example, via a display 316, such as a CRT or LCD monitor, and a light pen 318. The light pen 318 detects light emitted by the display 316 with a light sensor in the tip of the light pen 318. To select a particular screen or function, the operator touches a designated area of a screen on the display 316 and pushes the button on the light pen 318. Typically, the area touched changes color, or a new menu is displayed, confirming communication between the user and the controller 300.

The data signals received and evaluated by the controller 300 may be sent to a factory automation host computer 338. The factory automation host computer 338 may comprise a host software program 340 that evaluates data from several systems, platforms or chambers 100, and for batches of substrates 105 or over an extended period of time, to identify statistical process control parameters of (i) the processes conducted on the substrates 105, (ii) a property that may vary in a statistical relationship across a single substrate 105, or (iii) a property that may vary in a statistical relationship across a batch of substrates 105. The host software program 340 may also use the data for ongoing in-situ process evaluations or for the control of other process parameters. A suitable host software program comprises a WORKSTREAM™ software program available from aforementioned Applied Materials. The factory automation host computer 338 may be further adapted to provide instruction signals to (i) remove particular substrates 105 from the processing sequence, for example, if a substrate property is inadequate or does not fall within a statistically determined range of values, or if a process parameter deviates from an acceptable range; (ii) end processing in a particular chamber 100, or (iii) adjust process conditions upon a determination of an unsuitable property of the substrate 105 or process parameter. The factory automation host computer 338 may also provide the instruction signal at the beginning or end of processing of the substrate 105 in response to evaluation of the data by the host software program 340.

In one version, the controller 300 comprises a computer-readable program 320 that may be stored in the memory 308, for example on the non-removable storage media 312 or on the removable storage media 310. The computer readable program 320 generally comprises process control software comprising program code to operate the chamber 100 and its components, process monitoring software to monitor the processes being performed in the chamber 100, safety systems software, and other control software. The computer-readable program 320 may be written in any conventional computer-readable programming language, such as for example, assembly language, C++, Pascal, or Fortran. Suitable program code is entered into a single file, or multiple files, using a conventional text editor and stored or embodied in computer-usable medium of the memory 308. If the entered code text is in a high level language, the code is compiled, and the resultant compiler code is then linked with an object code of pre-compiled library routines. To execute the linked, compiled object code, the user invokes the object code, causing the CPU 306 to read and execute the code to perform the tasks identified in the program.

An illustrative block diagram of a hierarchical control structure of a specific embodiment of a computer readable program 320 is shown in FIG. 6 according to the present invention. Using the light pen interface 318, for example, an operator enters a process set and chamber number into the computer readable program 320 in response to menus or screens displayed on the display 318 that make up a process selector 321. The computer readable program 320 includes program code to control the substrate position, gas flow, gas pressure, temperature, RF power levels, and other parameters of a particular process, as well as code to monitor the chamber process. The process sets are predetermined groups of process parameters necessary to carry out specified processes. The process parameters are process conditions, including without limitations, gas composition, gas flow rates, temperature, pressure, and gas energizer settings such as RF or microwave power levels.

The process sequencer instruction set 322 comprises program code to accept a chamber type and set of process parameters from the computer readable program 320 or the process selector 321 and to control its operation. The sequencer instruction set 322 initiates execution of the process set by passing the particular process parameters to a chamber manager instruction set 324 that controls multiple processing tasks in the process chamber 100. The process chamber instruction set 324 may include, for example, a substrate positioning instruction set 326, a gas flow control instruction set 328, a gas pressure control instruction set 330, a temperature control instruction set 332, a gas energizer control instruction set 334, and a process monitoring instruction set 336. The substrate positioning instruction set 326 may comprise program code for controlling chamber components that are used to load the substrate 105 onto the support 150, and optionally, to lift the substrate 105 to a desired height in the chamber 100. The gas pressure control instruction set 330 comprises program code for controlling the pressure in the chamber 100 by regulating an open/close position of the gas flow valve 136 and/or the throttle valve 164. The temperature control instruction set 332 may comprise, for example, program code for controlling the temperature of the substrate 105 during processing. The gas energizer control instruction set 334 comprises program code for setting, for example, the RF power levels applied to the antenna 156 or the electrodes. The process monitoring instruction set 336 may comprise program code to monitor a process in the chamber 100. The gas flow control instruction set 328 comprises program code for controlling a flow rate of the process gas. For example, the gas flow control instruction set 328 may regulate an opening size of the gas flow valve 136 to obtain a desired gas flow rate from the gas distributor 130 into the chamber 100. In one version, the gas flow control instruction set 328 comprises program code to set a volumetric flow rate of the process gas introduced through the gas distributor 130. The velocity of the process gas ejected from the gas outlet 228 may be such that, when the ejected gas flow is obstructed, the thermal energy generated in the process gas selectively energizes a preselected component of the process gas.

In one version, the controller 300 controls the gas flow valve 136 to pulse the amount of flow of the process gas through the gas inlet 230 to selectively energize the process gas. For example, the gas flow valve 136 may periodically pulse the amount of flow of the process gas at a selected frequency to generate shockwaves to desirably dissociate the process gas. The pulsing frequency may correspond to, for example, a frequency of the shockwaves, which in turn affects certain characteristics of the energized process gas. For example, particular pulsing frequencies may accurately induce the generation of particular dissociated species.

While described as separate instruction sets for performing a set of tasks, it should be understood that each of these instruction sets can be integrated with one another, or the tasks of one set of program code integrated with the tasks of another to perform the desired set of tasks. Thus, the controller 300 and the computer program code described herein should not be limited to the specific version of the functional routines described herein; and any other set of routines or merged program code that perform equivalent sets of functions are also in the scope of the present invention. Also, while the controller is illustrated with respect to one version of the chamber 100, it may be used for any chamber described herein.

Although the present invention has been described in considerable detail with regard to certain preferred versions thereof, other versions are possible. For example, the substrate processing chamber of the present invention can be used for other processes, such as physical vapor deposition. Therefore, the appended claims should not be limited to the description of the preferred versions contained herein.

What is claimed is:

1. A substrate processing chamber comprising:
(a) a housing;
(b) a substrate support to support a substrate in the housing;
(c) a shockwave gas energizer to generate shockwaves in a process gas to at least partially energize the process gas, and provide the energized process gas into the housing to process the substrate; and
(d) a gas exhaust to exhaust the process gas from the housing.

2. A chamber according to claim 1 wherein the shockwave gas energizer comprises:
(i) a gas nozzle adapted to accelerate the process gas to a velocity of at least about Mach 1; and
(ii) a gas flow blocker to obstruct the accelerated flow of the process gas to generate shockwaves that at least partially energize the process gas.

3. A chamber according to claim 2 wherein the gas nozzle comprises:
a gas inlet to receive the process gas, the gas inlet comprising a first diameter;
a flow constricting throat comprising a second diameter; and
a gas outlet to eject the accelerated process gas, the gas outlet comprising a third diameter.

4. A chamber according to claim 3 wherein the gas nozzle comprises a wall that tapers radially inwardly from the gas inlet to the flow constricting throat and tapers radially outwardly from the flow constricting throat to the gas outlet.

5. A chamber according to claim 3 wherein the ratio of the first diameter to the second diameter is at least about 5:1.

6. A chamber according to claim 3 wherein the ratio of the third diameter to the second diameter is at least about 10:1.

7. A chamber according to claim 3 wherein the gas inlet is adapted to receive the process gas at a first pressure and the gas exhaust is adapted to maintain the process gas in the housing at a second pressure, and wherein the ratio of the first pressure to the second pressure is at least about 10:1.

8. A chamber according to claim 3 wherein the process gas flow ejected from the gas outlet has a principal flow direction, and wherein the gas flow blocker comprises a blocking surface that is perpendicular to the principal flow direction.

9. A chamber according to claim 3 comprising a controller to set a flow rate of the process gas flowing into the gas inlet of the gas nozzle so that the velocity of the process gas ejected from the gas outlet is such that, when the ejected gas flow is obstructed, the thermal energy generated in the process gas selectively energizes a preselected component of the process gas.

10. A chamber according to claim 2 wherein the gas nozzle is at a distance of less than about 1 cm from the gas flow blocker.

11. A chamber according to claim 2 wherein the shockwave gas energizer is mounted in the housing in opposing relationship to the substrate support.

12. A chamber according to claim 1 further comprising an electrical gas energizer comprising an inductive or capacitive energizer to further energize the process gas.

13. A substrate processing method comprising:
(a) placing a substrate in a process zone;
(b) generating shockwaves in a process gas to at least partially energize the process gas and providing the energized process gas to the process zone to process the substrate; and
(c) exhausting the process gas from the process zone.

14. A method according to claim 13 wherein (b) comprises:
(i) accelerating the process gas to a velocity of at least about Mach 1; and
(ii) obstructing the accelerated flow of the process gas to generate shockwaves that at least partially energize the process gas.

15. A method according to claim 14 comprising maintaining the process gas at a first pressure prior to step (i), and at a second pressure after step (ii), the ratio of the first pressure to the second pressure being at least about 10:1.

16. A method according to claim 15 wherein the ratio of the first pressure to the second pressure is at least about 2000:1.

17. A method according to claim 14 wherein the process gas is accelerated by constricting a flow of the process gas, and thereafter, expanding the flow of the process gas.

18. A method according to claim 17 comprising constricting the flow of the process gas by passing the process gas through a passageway that tapes from a first diameter to a second diameter, the ratio of the first diameter to the second diameter being at least about 5:1.

19. A method according to claim 18 comprising expanding the flow of the process gas by passing the process gas through a passageway that expands from the second diameter to a third diameter, the ratio of the third diameter to the second diameter being at least about 10:1.

20. A method according to claim 14 comprising accelerating the process gas flow to a predefined velocity so that when the accelerated process gas flow is obstructed, resultant shockwaves are generated that energize a preselected component the process gas.

21. A method according to claim 14 wherein the accelerated flow of the process gas traverses a distance of less than about 10 mm before being obstructed.

22. A method according to claim 13 further comprising electrically energizing the energized process gas by capacitively or inductively coupling energy to the process gas.

23. A substrate processing chamber comprising:
(a) a housing;
(b) a substrate support to support a substrate in the housing;
(c) a shockwave gas energizer comprising:
(i) a gas nozzle to provide an accelerated flow of process gas, the gas nozzle comprising a gas inlet to receive a process gas, a gas outlet to eject the process gas, and a flow constricting throat between the gas inlet and the gas outlet;
(ii) a gas flow blocker to obstruct the accelerated flow of the process gas to generate shockwaves in the process gas that at least partially energize the process gas; and
(iii) an aperture to provide the energized process gas into the housing; and
(d) a gas exhaust to exhaust the process gas from the housing.

24. A chamber according to claim 23 wherein the gas inlet has a first diameter, the flow constricting throat has a second diameter, and the ratio of the first diameter to the second diameter is at least about 5:1.

25. A chamber according to claim 23 wherein the gas outlet has a third diameter and the ratio of the third diameter to the second diameter is at least about 10:1.

26. A chamber according to claim 23 wherein the gas inlet is adapted to receive a process gas at a first pressure and the housing is adapted to contain a process gas at a second pressure, and wherein the ratio of the first pressure to the second pressure is at least about 10:1.

27. A substrate processing chamber comprising:
(a) a housing;
(b) a substrate support to support a substrate in the housing;
(c) means for generating shockwaves in a process gas to at least partially energize the process gas, and provide the energized process gas into the housing to process the substrate; and
(d) an exhaust to exhaust the process gas.

28. A chamber according to claim 27 wherein the means for generating shockwaves in the process gas comprises:
means for accelerating the process gas to a velocity of at least about Mach 1; and
means for obstructing the flow of the accelerated process gas.

29. A chamber according to claim 28 wherein the means for accelerating the process gas comprises means for constricting the flow of the process gas, and thereafter, expanding the flow of the process gas.

30. A substrate processing chamber comprising:
(a) a housing;
(b) a substrate support to support a substrate in the housing;
(c) two shockwave gas energizers positioned to face each other and direct an energized process gas toward a process zone between the two shockwave gas energizers, each shockwave gas energizer comprising:
(i) a gas nozzle to provide an accelerated flow of process gas, the gas nozzle comprising a gas inlet to receive a process gas, a gas outlet to eject the process gas, and a flow constricting throat between the gas inlet and the gas outlet;
(ii) a gas flow blocker to obstruct the accelerated flow of the process gas to generate shockwaves in the process gas that at least partially energize the process gas;
(iii) a gas guide extending below the gas flow blocker such that solid particles from the gas nozzle fall down onto the gas guide rather than onto the substrate; and
(iv) an aperture to provide the energized process gas into the housing; and
(d) a gas exhaust to exhaust the process gas from the housing.

31. A chamber according to claim 30 wherein one of the gas inlets has a first diameter, the flow constricting throat has a second diameter, and the ratio of the first diameter to the second diameter is at least about 5:1.

32. A chamber according to claim 30 wherein one of the gas outlets has a third diameter and the ratio of the third diameter to the second diameter is at least about 10:1.

33. A chamber according to claim 30 wherein one of the gas inlets is adapted to receive a process gas at a first pressure and the housing is adapted to contain a process gas at a second pressure, and wherein the ratio of the first pressure to the second pressure is at least about 10:1.
